(19) United States
(12) Patent Application Publication
(10) Pub. No.: US 2017/0016814 A1
(43) Pub. Date: Jan. 19, 2017

(54) BIOMOLECULE ANALYSIS DEVICE

(71) Applicant: HITACHI, LTD.

(72) Inventors: Masataka Shirai, Tokyo (JP); Hideki Kambara, Tokyo (JP); Tomoharu Kajiyama, Tokyo (JP)

(73) Assignee: HITACHI, LTD., Tokyo (JP)

(21) Appl. No.: 15/107,184

(22) PCT Filed: Dec. 27, 2013
(86) PCT No.: PCT/JP2013/085093

§ 371 Date: Jun. 22, 2016

(51) Int. Cl. G01N 15/14; G02B 21/16; G01N 1/44; G01N 21/64; G01N 33/52; C12Q 1/68

(52) U.S. Cl. CPC G01N 15/1475; G01N 33/52; C12Q 1/6837; G01N 1/44; G01N 21/6428; G01N 21/6458; G02B 21/16; G01N 2015/1006

(57) ABSTRACT

An object is to provide a biomolecule analyzer capable of collecting and analyzing a biomolecule in a single cell without damaging neighboring cells.

In order to achieve this object, a biomolecule analyzer according to the present invention is characterized by including a unit which obtains an optical image of a plurality of cells, a unit which disrupts a part or the whole of at least one cell of the plurality of cells, an array device in which regions for capturing a biomolecule in the cell released by the disrupting unit are arranged, and a unit which associates the region in which the biomolecule is captured in the array device with a portion corresponding to the cell disrupted by the disrupting unit in the optical image.

### CROSS REFERENCE TO PRIOR APPLICATIONS

This application is a U.S. National Phase application under 35 U.S.C. §371 of International Application No. PCT/JP2013/085093, filed on Dec. 27, 2013. The International Application was published in Japanese on Jul. 2, 2015 as WO 2015/097858 A1 under PCT Article 21(2). The contents of the above applications are hereby incorporated by reference.

### SEQUENCE LISTING

The instant application contains a Sequence Listing which has been submitted electronically in ASCII format and is hereby incorporated by reference in its entirety. Pursuant to 37 C.F.R. §1.52(e) (5), the Sequence Listing text file, identified as 072388_1261_Sequence_Listing_.txt, is 4,796 bytes and was created on Jun. 15, 2016. The Sequence Listing, electronically filed herewith, does not extend beyond the scope of the specification and thus does not contain new matter.

### TECHNICAL FIELD

The present invention relates to a biomolecule analyzer.

### BACKGROUND ART

Recently, the importance of a single-cell analysis in which an analysis is performed by paying attention to a difference in the genome, gene expression, or protein among individual cells when a genome analysis, a gene expression analysis, or a protein analysis is performed for a biological tissue composed of a large number of cells has started to be recognized. In a conventional analysis, DNA, RNA, or protein was extracted from a large number of cells (10³ to 10⁶ or more cells) sampled from a biological tissue as one type of sample, and the analysis was performed, and therefore, an average analysis in the sample was performed. Due to this, even if the existence amount in the individual cells of the DNA, RNA, or protein deviated from the average value, it was difficult to make an evaluation. The single-cell analysis is important as an analysis method for solving such a problem of averaging. In particular, in a study of cancer or iPSC (induced pluripotent stem cells), it is known that the behavior of a small number of cells is quite different from the average behavior of a biological tissue, and the importance of a single-cell analysis has been pointed out.

In a bulk analysis in which the average behavior of a large number of cells sampled from a biological tissue, the information regarding extremely various types of DNA, RNA, or protein can be obtained by one measurement. However, such a bulk analysis method cannot be simply applied to a single-cell analysis. In many bulk analysis methods, the minimum necessary sample size is 1,000 times to 1,000,000 times that at the single-cell level, and the method lacks necessary sensitivity in many cases. Then, in order to realize a single-cell analysis, it is necessary to isolate a single cell in the first place.

In the case of a tissue section or adherent culture cells, it is possible to isolate cells in principle by subjecting the cells to a chemical treatment such as a trypsin treatment thereby cleaving the bond between the cells.

Further, as described in NPL 1 and NPL 2, it is possible to dissect a specific cell group on a microscopic image by using a technique called laser microdissection or laser capture microdissection.

### CITATION LIST

### Non Patent Literature

NPL 1: BioTechniques 27, 2: 362-367 (August 1999)

NPL 2: Cellular and Molecular Biology 44 (5), 735-746 (1998)

### SUMMARY OF INVENTION

### Technical Problem

In the case where a bond between cells is cleaved by a chemical treatment such as a trypsin treatment as in the conventional method, the cells in a tissue fall apart and also due to the chemical treatment for isolation, a cell state, that is, the gene expression level, the protein level, etc. may be changed, which was also the matter of great concern.

Further, in laser microdissection or laser capture microdissection as described in NPL 1 and NPL 2, in order to dissect a tissue section with a thickness which is equal to or larger than at least the size of a cell using a laser, a dissection allowance of several micrometers or more is needed. This dissection allowance of several micrometers or more is comparable to the size of a cell. Therefore, in the case where the cells were in proximity to each other, it was difficult to isolate the cell without damaging the cells in proximity to each other. Further, there was also a problem that by disrupting neighboring cells, a biomolecule contained in the neighboring cells is mixed in a sample solution to decrease the measurement accuracy. In addition, the isolation of cells from a section composed of a plurality of layers could not be performed by a conventional technique because the conventional technique is a technique of performing dissection in the two-dimensional plane.

In view of this, an object of the present invention is to provide a biomolecule analyzer capable of collecting and analyzing a biomolecule in a single cell without damaging neighboring cells.

### Solution to Problem

In order to achieve the above object, the biomolecule analyzer of the present invention is characterized by including a unit which obtains an optical image of a plurality of cells, a unit which disrupts a part or the whole of at least one cell of the plurality of cells, an array device in which regions for capturing a biomolecule in the cell released by the disrupting unit are arranged, and a unit which associates the region in which the biomolecule is captured in the array device with a portion corresponding to the cell disrupted by the disrupting unit in the optical image.

### Advantageous Effects of Invention

According to the present invention, a biomolecule such as DNA, RNA, or protein in adherent culture cells or cells in a tissue section is collected in the array device for each single cell, and a region in which the biomolecule is collected on the array device is associated with the cell on an optical image obtained by a microscope, whereby the biomolecule in the single cell can be analyzed without damaging neighboring cells while avoiding the contamination with the biomolecule from another cell. Other objects, configurations, and advantageous effects will become apparent from the following description of the embodiments.

### BRIEF DESCRIPTION OF DRAWINGS

FIG. 1 is a structural view of a biomolecule analyzer according to a first embodiment of the present invention.

FIG. 2 is a top view of a pore array sheet in the first embodiment of the present invention.

FIG. 3 is a view showing the structure of an electrode in the case where a biomolecule is subjected to dielectrophoresis.

FIG. 4 is a flowchart for illustrating the operation of the biomolecule analyzer according to the first embodiment of the present invention.

FIG. 5 is a view for illustrating a sample preparation method (first half) in the first embodiment of the present invention.

FIG. 6 is a view for illustrating a sample preparation method (latter half) in the first embodiment of the present invention.

FIG. 7 is a view for illustrating the function of a tag sequence for molecule discrimination.

FIG. 8 is a view for illustrating a case where sample preparation is performed by dividing an array device.

FIG. 9 is a structural view in the case where a differential interference contrast microscope is used as a microscope system.

FIG. 10 is a structural view in the case where a CARS microscope is used as a microscope system.

FIG. 11 is a flowchart showing the procedure of a gene expression data analysis.

FIG. 12 is a view obtained by plotting the result of a principal factor analysis.

FIG. 13 is a view for illustrating a sample preparation method (1) in a second embodiment of the present invention.

FIG. 14 is a view for illustrating a sample preparation method (2) in the second embodiment of the present invention.

FIG. 15 is a view for illustrating a sample preparation method (3) in the second embodiment of the present invention.

FIG. 16 is a structural view of a biomolecule analyzer according to a third embodiment of the present invention.

FIG. 17 is a view showing a bead array in a fifth embodiment of the present invention.

### DESCRIPTION OF EMBODIMENTS

Hereinafter, the present invention will be described in detail.

In the present invention, a part of an intracellular tissue (a cell membrane or the like) corresponding to an optical image of an adherent culture cell or a tissue section on a plate is disrupted by abrasion or the like using a laser, and a biomolecule to be measured among the biomolecules released from the disrupted cell is captured in a specific region of an array device. More generally, the biomolecule analyzer of the present invention includes a unit which obtains an optical image of a plurality of cells disposed on a plane, a unit which disrupts apart or the whole of at least one cell of the plurality of cells by convergent laser irradiation, convergent sound wave irradiation, needle perforation, hollow needle perforation, or the like, a unit (array device) which captures the biomolecule released to the outside of the cell by disruption for each cell in regions arranged in an array, and a unit which associates the region on the array device with the cell on the optical image.

That is, instead of performing isolation by dissection of cells, a part of an intracellular tissue such as a cell membrane is disrupted without isolating cells, and therefore, even if how a cell to be measured is adhered to neighboring cells, a part or the whole of the cell can be disrupted with high resolution obtained by a light collecting ability of a laser or the like without damaging the neighboring cells, and thus, a biomolecule in the cell can be collected.

Further, only a biomolecule of interest is captured using the array device without collecting the whole cells simultaneously with the neighboring cellular tissues, and therefore, in the subsequent sampling treatment, contamination with impurities other than the biomolecule of interest can be prevented, and thus, a highly accurate analysis can be achieved. In addition, the use of a reagent for an unnecessary sampling treatment for biomolecules other than the biomolecule of interest is no longer needed, and therefore, the cost for the analysis can be reduced.

According to the present invention, with respect to the optical image obtained by a fluorescence microscope, Raman microscope, or the like without disrupting cells, the quantitative information of a biomolecule such as a gene or a protein obtained by disrupting cells can be associated. This association enables the evaluation of detailed and dynamic characteristics of cells by associating data obtained by an imaging unit with which it is difficult to quantitatively evaluate a large number of types of biomolecules, but the cell can be measured alive with data obtained by a unit with which detailed quantitative data regarding the biomolecule can be obtained, but the dynamic characteristics of the cell cannot be evaluated because the cell is disrupted.

In the past, in order to analyze a large number of cells at a time at low cost, the present inventors developed a device for performing an analysis of gene expression in a large number of cells by constructing a cDNA library utilizing a porous membrane or the like and obtaining the two-dimensional distribution of gene expression (US Patent Application Publication No. 2012/0245053).

In a method using this conventional device, mRNA extracted from a cell observed by a microscope is captured on the device immediately below the cell, and then reverse transcription is performed, whereby an analysis of gene expression in the cell corresponding to the microscopic image of the cell can be performed without isolating the cell. Therefore, a problem that the association between the microscopic image of the cell at the time of isolation of the cell and the cell to be analyzed for gene expression is lost is avoided.

However, in the above conventional device, mRNA is captured on the device composed of a porous membrane immediately below the cell, and therefore, the device has a problem that if cells are overlapped in the axial direction perpendicular to the plane of the device, a cell in which the extracted mRNA is originally present cannot be specified. In addition, it was necessary to perform extraction and capture of a biomolecule such as mRNA from all the cells once on the device composed of a porous membrane, and thereafter perform a sample treatment for the subsequent analysis for all the cells even if there were a large number of cells for which it was not necessary to perform a detailed genome analysis, gene expression analysis, or proteome analysis at the time of optical imaging by a microscope. Therefore, the device also had a problem that a reagent for sample preparation is wasted. In the present invention, a single cell is disrupted by a laser or the like, and only a biomolecule in the cell can be analyzed, and therefore, the above problems can be solved.

Further, in the case where a cell sample placed on the above-mentioned conventional device composed of a porous membrane is subjected to optical imaging using a transmission microscope such as a differential interference contrast microscope or a nonlinear Raman microscope, there was a problem that light scattering occurs on the device to decrease the resolution of imaging. Also this problem can be solved by the present invention. That is, in the present invention, it is not necessary to perform microscopic observation in a state where cells are placed on the device, and therefore, in the case where a transmission microscope is used, the decrease in the resolution of an optical image due to light scattering can be avoided.

Further, in the case where an analysis of adherent culture cells is performed using the above-mentioned conventional device composed of a porous membrane, there was a problem that a material to which the culture cells are adhered is limited to a material composed of the porous membrane. The present invention is configured such that after an optical image is obtained by a microscope, the array device is brought close to a cell of interest, the cell is disrupted by a laser or the like, and a biomolecule in the cell is collected for each single cell, and therefore, a single-cell analysis can be performed by using a common plate to be used for adherent culture cells.

Hereinafter, the present invention will be described in further detail based on embodiments, however, the present invention is by no means limited thereto.

### First Embodiment

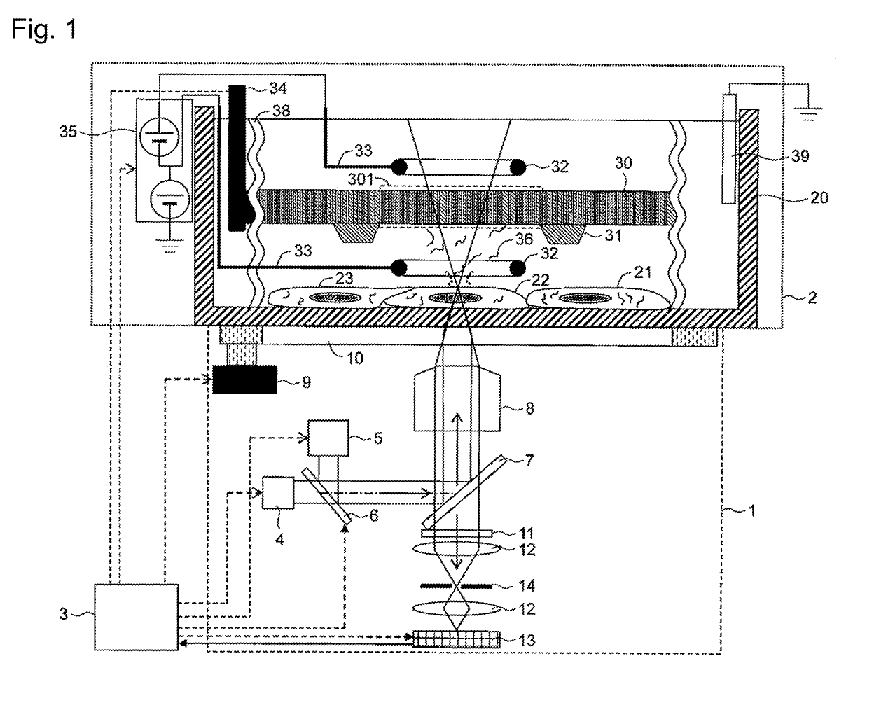

The biomolecule analyzer according to this embodiment includes a unit which obtains an optical image of a sample composed of adherent culture cells using a laser fluorescence microscope and a unit which disrupts the cell using a laser light source, and is a device capable of performing a gene expression analysis by capturing mRNA in the cell in an array device. In FIG. 1, the structural view of the biomolecule analyzer according to this embodiment is shown. This biomolecule analyzer is constructed from a microscope system 1 which has a function of obtaining an optical image and disrupting a part or the whole of the cell at a given position on the optical image, an array device in which regions for capturing a biomolecule (mRNA) released or diffused from the cell are arranged, a biomolecule collection system 2 which has a mechanism for driving the same and a mechanism for moving a sample cell, and a control system 3 which controls the movement of these two systems.

The microscope system 1 includes a laser light source for fluorescence microscope 4. In this embodiment, as the laser light source for fluorescence microscope 4, a semiconductor laser with an output power of 50 mW and a continuous oscillation of 488 nm is used. Other than this, a laser with a wavelength of 405 nm or 633 nm may be used according to the variation of fluorescence desired to be observed, or it is also possible to output laser light with a plurality of wavelengths from the laser light source for fluorescence microscope 4 by using a dichroic mirror or an optical filter.

Further, the microscope system 1 includes a laser light source for cell disruption 5. In this embodiment, as the laser light source for cell disruption 5, a pulse laser with a 355 nm band (maximum average output power: 2 W, repetition frequency: 5 kHz) is used. The laser light source for fluorescence microscope 4 and the laser light source for cell disruption 5 perform multiplexing using a dichroic mirror 6 (edge wavelength: 409 nm). Incidentally, the laser light source for cell disruption 5 may be used as a light source for fluorescence microscope. In fact, in this embodiment, the laser light source for cell disruption 5 is used for observation of a nucleus.

As a fluorescent dye for dyeing cells, Annexin VFITC and Hoechst Dye are used in this embodiment. The former emits light at 535 nm by excitation at 488 nm, and the latter emits light at 465 nm by excitation at 355 nm. A cell membrane in the course of apoptosis is dyed with the former fluorescent dye, and a nucleus is dyed with the latter fluorescent dye.

As a dichroic mirror 7, for example, a dichroic mirror with an edge wavelength of 505 nm is used when performing FITC fluorescence imaging, and a dichroic mirror with an edge wavelength of 385 nm is used when performing Hoechst fluorescence imaging.

The microscope system 1 includes an objective lens 8. As the objective lens 8, for example, an objective lens with an NA of 0.8 and a magnification of 40 times is used.

The optical imaging of cells can be performed as follows. First, on a plate 20 having a bottom face with a cover glass thickness (0.18 mm) which is transparent and does not emit fluorescence, cultured adherent culture cells 21, 22, and 23 are placed, and the plate 20 is placed on a sample stage 10 provided with an open through-hole for light path in a central portion.

The sample stage 10 is driven at a pitch of 50 nm by a stage 9 movable in the X, Y, and Z directions. A fluorescence light receiving system is composed of a band-pass filter 11 which removes light with wavelengths other than fluorescence, an imaging lens 12, a PMT (photomultiplier tube) serving as an optical receiver 13, and a pinhole 14. By synchronously driving these members, an optical image of a plurality of cells is obtained. That is, a fluorescence signal from one point at which excitation light is collected is obtained by the optical receiver 13 while driving the XYZ stage 9, and an optical image is formed in a control system.

The biomolecule collection system 2 includes the array device in which regions for capturing a biomolecule such as mRNA released from the cell are arranged. For example, mRNA is captured in a plurality of regions of the array device for each single cell, and then, a reverse transcription reaction is performed in the array device, whereby a cDNA library can be constructed. In this embodiment, the array device is constructed from a porous membrane which is transparent and has a large number of through-holes formed perpendicularly to the plane, and is hereinafter referred to as “pore array sheet 30”. Further, a member in which a cDNA library is formed in the pore array sheet 30 is referred to as “cDNA library pore array sheet”.

In this embodiment, as the pore array sheet 30, a porous membrane made of aluminum oxide with a thickness of 80 μm and a size of 2 mm×2 mm and having a large number of through-holes with a diameter of 0.2 μm formed by anodization is used. In the pore array sheet 30, a separation wall 31 for separating the regions for capturing the biomolecule from each other can be formed. This separation wall 31 can be formed by a semiconductor process using polydimethylsiloxane (PDMS), and has a thickness of about 80 μm, and can be brought into close contact with the pore array sheet 30.

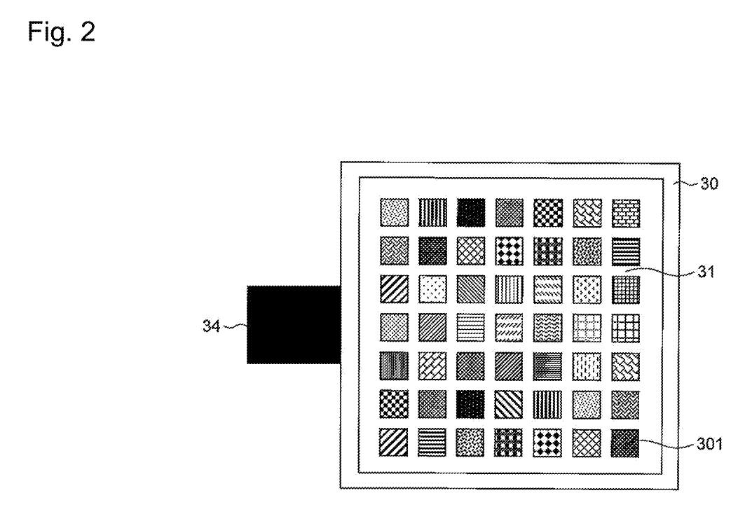

A top view of the pore array sheet 30 is shown in FIG. 2. In the pore array sheet 30 (size: 2 mm×2 mm, thickness: pi), a large number of regions 301 for capturing a biomolecule, for example, mRNA are formed. Here, the size of the region 301 is set such that one side length is 100 μm, and an interval is 80 μm (arranged at a pitch of 180 μm). The size of the region 301 can be designed freely from about 1 μm to about 10 mm according to the amount of the biomolecule to be captured or the ease of diffusion thereof in the plane (the size of the molecule).

As the array device, other than the pore array sheet 30 composed of a porous membrane formed by anodization of aluminum, a member having a large number of through-holes formed by anodization of a material such as silicon may be used. In addition, the array device may be constructed by providing a large number of through-holes in a silicon oxide thin film or a silicon nitride thin film using a semiconductor process. Further, as will be described later, a pore array sheet may be formed by packing beads with various sizes in box-shaped portions, or a film in which a monolithic column to be used as a column for liquid phase chromatography is formed thin can also be used as the array device. Further, membranes of various materials, that is, a cellulose membrane, a glass fiber membrane, a track-etched membrane, a nylon membrane, a polypropylene membrane, a PTFE membrane, and the like may be used. At this time, the separation wall 31 can be formed using other than the PDMS resin, a semiconductor material such as silicon or another resin material by combining known semiconductor processes. The present invention relates to the biomolecule analyzer incorporating the array device as described above therein, however, other than this, the above-mentioned array device, in which a plurality of regions capable of being associated with the optical image of a plurality of cells are arranged and formed, itself is provided as a kit for a biomolecule analysis. By using the kit of this array device in combination with the unit which disrupts a cell with a laser or the like by a user, an analysis of a biomolecule of a single cell can be efficiently performed.

As shown in FIG. 1, as a unit which guides the biomolecule released from the cell to a specific region in the pore array sheet 30 by electrophoresis, a loop-shaped platinum electrode 32 is connected to the tip end of a shield wire 33. The diameter of a wire material of the platinum electrode 32 is 30 μm, and the wire material is folded into two, and the portion where a lead wire is connected is twisted into one, and then, the wire material is processed into a circle with a diameter of 100 μm on the loop side. Two such electrodes are formed and disposed so as to sandwich the pore array sheet 30, and a direct current of 1.5 V is applied thereto by a power supply 35. The released mRNA 36 has a negative charge, and therefore, the platinum electrode 32 on the upper side is used as a positive electrode. However, a silver-silver chloride reference electrode 39 is provided, and a voltage of 0.2 V is applied to the platinum electrode 32 on the lower side. By such an operation, the mRNA 36 can be guided to the inside of the region 301 for capturing the biomolecule by electrophoresis. In addition, in order to realize the concentration of mRNA by electrophoresis in the lateral direction for further improving the efficiency of capturing the biomolecule, the diameter of the loop of the platinum electrode 32 on the upper side may be set to 50 μm. In this case, the diameter of the wire material is set to 10 μm.

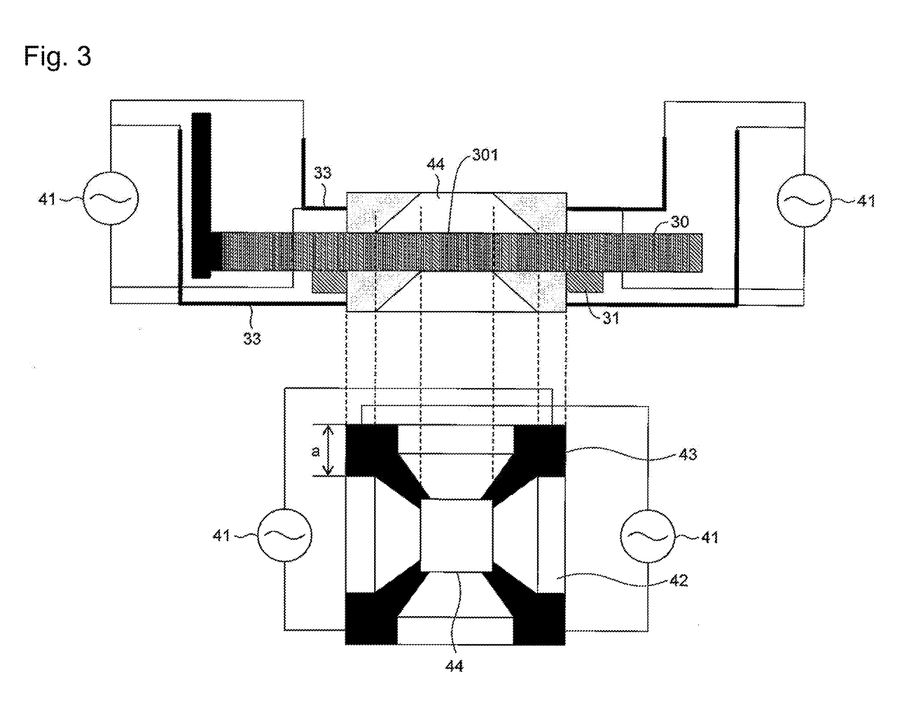

The structure of the electrode to be used for electrophoresis and the method for applying a voltage are not limited to the above-mentioned method. For example, in order to perform dielectrophoresis of a microgranule such as an exosome or a DNA fragment of 40 kb or more, and a protein of 10 kDa or more, an electrode structure as shown in FIG. 3 may be used. That is, in the center of a quartz substrate 42 in the form of a square with a thickness of 200 μm, a through-hole 44 is formed by wet etching, and four gold electrodes 43 are formed on an inner wall and an outer wall of the quartz substrate 42, whereby a quadrupole terminal is formed. The thickness of the gold electrode 43 is set to 1 μm and the width thereof is set to 40 μm (a portion a). By applying an alternating electric field with an amplitude of 10 V and a frequency of kHz by a power supply 41, the above-mentioned large biomolecule or granule can be guided into the region 301 by dielectrophoresis.

By capturing a peptide or a protein extracted from a single cell in the array device once, the measurement sensitivity when performing a mass analysis thereafter can be increased. That is, in a mass analysis of a tissue section using MALDI, it is necessary to mix a chemical substance called “matrix” with a sample in an appropriate ratio and perform irradiation with a laser. When the ratio is not appropriate, the efficiency of ionization of a molecule of interest is largely decreased. However, in MALDI for a tissue section, a matrix material is merely added from the surface, and therefore, a uniform material in an appropriate ratio cannot be obtained. Due to this, the ionization efficiency generally varies depending on the position and also decreases. However, by adsorbing and capturing a biomolecule of interest for each single cell, and thereafter performing MALDI for the array device having the molecule of interest adsorbed thereon, a mass analysis of a peptide or a protein in the cell in the tissue section can be performed with high efficiency. In addition, since only the biomolecule of interest is selectively captured, the ion suppression effect of impurities is also decreased, and thus, the ionization efficiency is further improved.

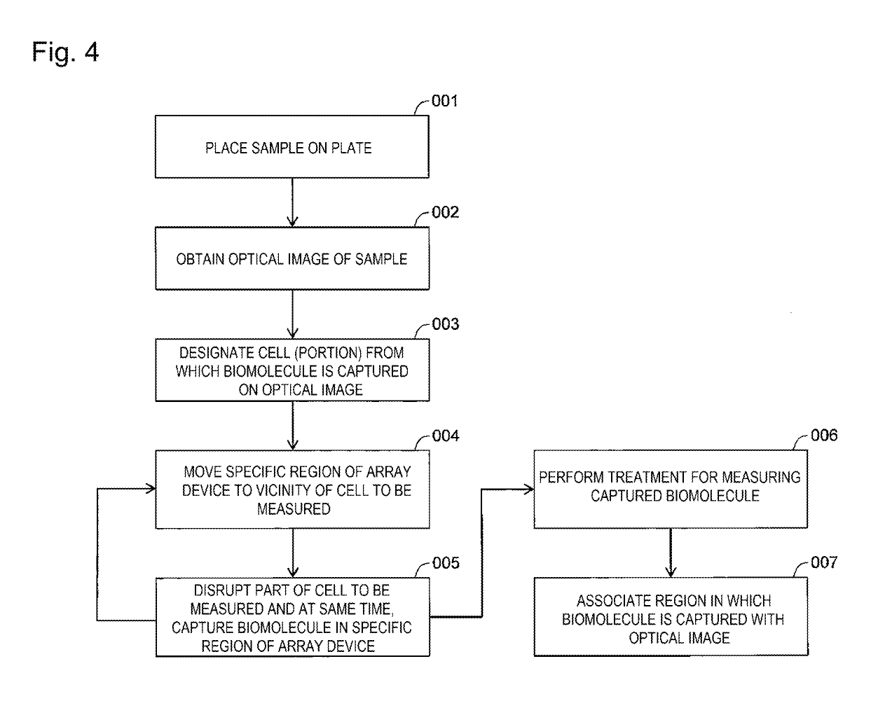

Next, an operation flow of the biomolecule analyzer according to this embodiment will be described. In FIG. 4, a flowchart is shown. First, a sample including adherent culture cells 21, 22, and 23 are placed on the plate 20 (step 001). In this embodiment, the measurement target is culture cells, and therefore, the cells are cultured beforehand using the plate 20 and the cells as the measurement target are adhered to the bottom face. In the case where the sample is a frozen section, this frozen section is placed on the plate 20. Alternatively, a material obtained by three-dimensionally arranging a plurality of cells in a gel may be used as the sample. Subsequently, by using the microscope system 1, an optical image of a target cell group is obtained (step 002), a biomolecule is collected, and a user determines a target cell to be measured (step 003). Subsequently, in the control system 3, the user designates the cell or a portion of the cell as the measurement target. In general, it is often the case that the user designates a plurality of cells as the measurement target. In this case, the control system 3 determines the order of the cells from which the biomolecule is captured, and first, a specific region (for example, the region 301 at the first row and first column (1,1)) of the pore array sheet 30 is brought close to the vicinity (immediately above the cell in the example in FIG. 1) of the cell to be measured first using an XYZ stage 34 (step 004). In this embodiment, the distance between the lower surface of the pore array sheet 30 and the plate 20 is set to 300 μm, however, this distance can be changed depending on the type of the biomolecule to be collected or the structure of the electrode. For example, the distance is preferably from 1 μm to about 10 mm. The movement of the pore array sheet 30 by the XYZ stage 34 is automatically performed by the control system 3 according to a previously installed program.

After the control system 3 confirms the completion of the movement, a voltage is applied to the platinum electrode 32 for electrophoresis, and at the same time, in order to disrupt the cell membrane of the cell to be measured, the cell is irradiated with laser light from the laser light source for cell disruption 5 (step 005). Here, the irradiation time can be set to, for example, 10 seconds, and the time for driving electrophoresis can be set to 60 seconds.

After completion of disruption of one cell and capture of the biomolecule in the cell, a specific region (for example, the region 301 at the first row and second column (1,2)) of the pore array sheet 30 is brought close to the vicinity (immediately above the cell in the configuration in FIG. 1) of the cell having been registered in the control system 3 and to be measured subsequently (step 004). The cell having been registered in the control system 3 and to be measured subsequently is irradiated with laser light from the laser light source for cell disruption 5 (step 005). At this time, in the same manner as described above, a voltage is applied to the platinum electrode 32 simultaneously with the irradiation. Thereafter, the measurement target is shifted sequentially to the designated cell, and the cell is disrupted, the biomolecule in the cell is captured in the specific region 301 of the pore array sheet 30, and then, the treatment for measuring the captured biomolecule is performed (step 006). Finally, a portion corresponding to the disrupted cell in the optical image and the region 301 in which the biomolecule is captured in the pore array sheet 30 are associated with each other and shown to the user (step 007).

Here, the number of cells to be disrupted is set to one, however, in the case where coarser resolution data is desired to be obtained, with respect to one region 301 on the array device, mRNA released when a plurality of cells are disrupted and subjected to electrophoresis may be captured. As for the disruption at this time, a plurality of cells may be disrupted simultaneously, or a plurality of cells may be sequentially disrupted one by one without moving the array device.

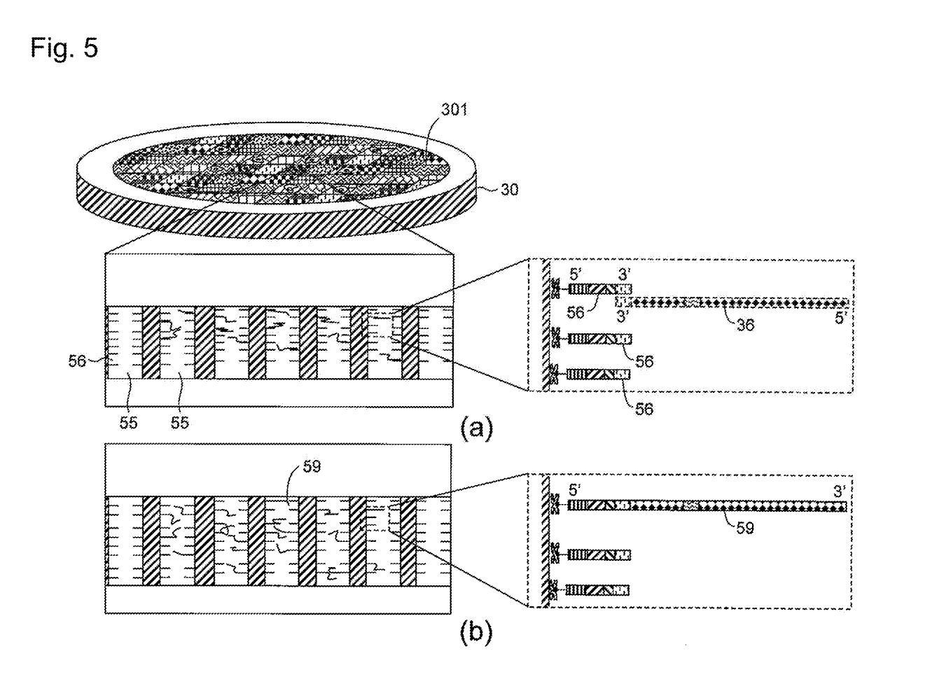

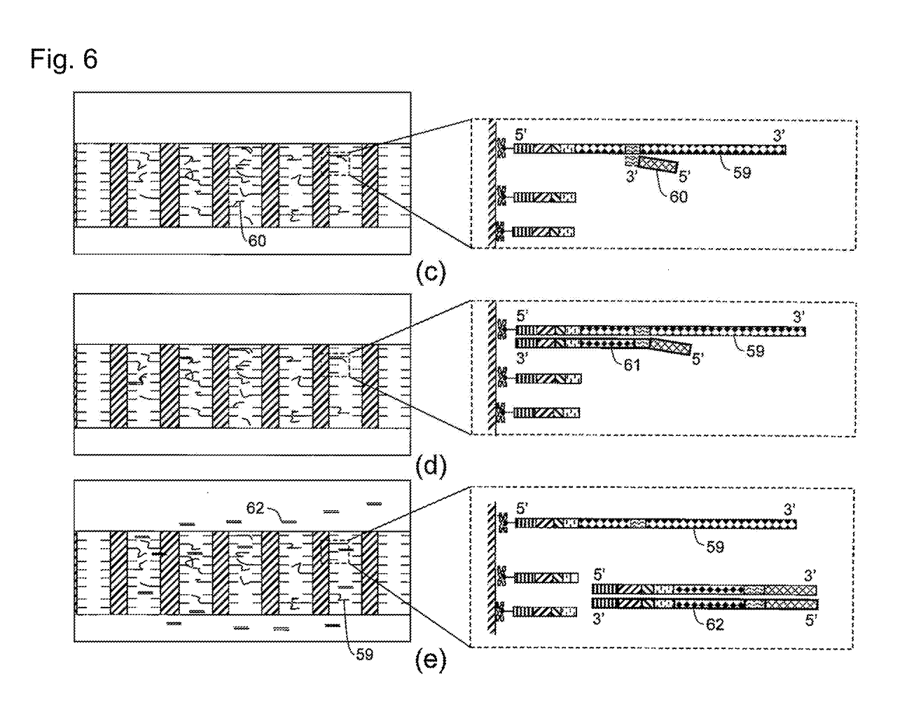

Next, a sample preparation method for obtaining gene expression analysis data for each cell from the mRNA captured by the pore array sheet 30 will be described with reference to FIGS. 5 and 6. In this embodiment, the pore array sheet 30 is a chip with a size of 2 mm×2 mm, and a gene expression analysis is performed by detaching the pore array sheet 30 from the XYZ stage 34, and performing a treatment as described below in a reaction vessel (tube). This method includes capturing the mRNA on a DNA probe immobilized on the inside of the pore array sheet 30 (FIG. 5(a)), preparing a cDNA library by reverse transcription (synthesis of a first strand) in the inside of the pore array sheet 30 (FIG. 5(b)), synthesizing a second strand thereafter (FIGS. 6(c) and 6(d)), and performing PCR amplification (FIG. 6(e)). The reverse transcription is performed by using the information of the position of the cell in which the captured mRNA is originally present as the sequence information, and therefore, a DNA probe 56 immobilized on the inside of the pore array sheet 30 includes a cell recognition sequence as a sequence which is different for each position in the pore array sheet 30, and the mRNA 36 is captured by this DNA probe 56. In this embodiment, the cell recognition sequence which is different for each region 301 in the pore array sheet 30 in FIG. 5(a) is immobilized. This DNA probe 56 for capturing mRNA includes a common sequence for PCR amplification (Forward direction), a tag sequence for cell discrimination, a tag sequence for molecule discrimination, and an oligo (dT) sequence from the 5′ end side. By introducing the common sequence for PCR amplification into the DNA probe 56, in the subsequent PCR amplification step, this sequence can be used as a common primer. Further, by introducing the tag sequence for cell discrimination (for examples, 5 bases) into the DNA probe 56, 4⁵=1024 single cells can be recognized. That is, a cDNA library can be prepared from 1024 single cells at a time, and therefore, an effect that the cost of reagents and the labor can be reduced to 1/1024 is obtained, and also it becomes possible to recognize as to which cell the sequence data finally obtained using a sequencer is derived from. Further, by introducing the tag sequence for molecule discrimination (for examples, 15 bases) into the DNA probe 56, 4¹⁵=1.1×10⁹ types of molecules can be recognized, and therefore, it is possible to recognize as to which molecule the enormous decoded data obtained using a sequencer is derived from. That is, amplification bias among genes that occurs in the amplification step can be corrected, and therefore, it becomes possible to quantitatively determine the level of mRNA originally present in the cell with high accuracy. The oligo (dT) sequence located closest to the 3′ end is hybridized to the poly A tail added to the 3′ end side of the mRNA, and therefore, utilized for capturing the mRNA (FIG. 5(a)).

In this embodiment, in order to capture mRNA, a poly T sequence is used as a part of the DNA probe 56, however, it goes without saying that in order to perform a microRNA analysis or a genome analysis, a part of a complementary sequence to the sequence desired to be analyzed may be used in place of the poly T sequence.

Next, a method for preparing the cDNA library pore array sheet will be described. A through-hole 55 formed in the pore array sheet 30 goes through the pore array sheet 30 in the thickness direction of the pore array sheet 30, and the through-holes 55 are completely mutually independent. The surface of the inner wall of the through-hole 55 is hydrophilic, and very few proteins are adsorbed on the surface, and thus, an enzymatic reaction efficiently proceeds. First, a silane coupling treatment or the like is performed for the surface of the pore array sheet 30, and the DNA probe 56 is immobilized on the surface of the through-hole 55. Since the DNA probe 56 is immobilized on the surface in a ratio of, for example, one per 30 to 100 nm² in average, 4 to 10×10⁶ DNA probes 56 are immobilized per through-hole 55. Subsequently, in order to prevent surface adsorption, the surface is coated with a surface coating agent. The surface coating may be performed simultaneously with immobilization of the DNA probe. The density of the DNA probe 56 is set to a density capable of capturing mRNA passing through this space with an efficiency of almost 100%.

Next, a method for preparing a cDNA library in the pore array sheet 30 by capturing mRNA 36 from a cell in the inside of the pore array sheet 30 will be described. As described above, the mRNA 36 which is released from a cell disrupted by irradiation with a laser and is negatively charged is subjected to electrophoresis by the platinum electrode 32, and guided to the inside of the through-hole 55 of the pore array sheet 30. As shown in FIG. 5(a), in the through-hole 55, the poly A tail of the mRNA 36 is captured by the oligo(dT) portion of the DNA probe 56. Then, a first strand cDNA strand 59 is synthesized by using the mRNA 36 captured by the DNA probe 56 as a template. In this step, for example, the through-hole 55 is filled with a solution containing a reverse transcriptase and a synthetic substrate, and a complementary strand synthesis reaction is performed for about 50 minutes by gradually increasing the temperature to 50° C. (FIG. 5(b)). After completion of the reaction, RNase is made to flow through the through-hole 55 to degrade and remove the mRNA 36. Then, a liquid containing an alkaline denaturant and a washing liquid are made to flow through the through-hole 55 to remove the residue and the degraded products. By the process described up to this point, a cDNA library as shown in FIG. 5(b) reflecting the original position of the cell is constructed in the through-hole 55. Subsequently, a plurality (up to 100 types) of target gene-specific sequence primers 60 to which a common sequence for PCR amplification (Reverse) has been added are annealed to the first strand cDNA strand 59 (FIG. 6(c)), and by a complementary strand elongation reaction, a second strand cDNA strand 61 is synthesized (FIG. 6(d)). That is, the second strand cDNA strand is synthesized under multiplex conditions. By doing this, with respect to each of the plurality of target genes, double-stranded cDNA 62 having the common sequences for amplification (Forward/Reverse) at both ends, and containing the tag sequence for cell discrimination, the tag sequence for molecule discrimination, and the gene-specific sequence is synthesized. Further, in this embodiment, as the gene-specific sequences, 20 types of sequences (ATP5B, GAPDH, GUSB, HMBS, HPRT1, RPL4, RPLP1, RPS18, RPL13A, RPS20, ALDOA, B2M, EEF1G, SDHA, TBP, VIM, RPLP0, RPLP2, RPLP27, and OAZ1, SEQ ID NOS: 3 to 22) corresponding to sequences composed of 20±5 bases in a region 109±8-bases upstream from the poly A tail of the target genes are used. This is because the PCR amplification products are made to have the same size of about 200 bases in the subsequence PCR amplification step. By making the amplification products have the same size, an effect that a complicated size fractionation purification step (electrophoresis→cutting out of gel→extraction and purification of PCR amplification products) can be omitted and the PCR amplification products can be directly used for parallel amplification (emulsion PCR or the like) from one molecule is obtained. Subsequently, PCR amplification is performed using the common sequences for amplification (Forward/Reverse), whereby PCR amplification products derived from the plurality of genes are prepared (FIG. 6(e)). Conventionally, there has been known a problem of so-called amplification bias in which the amplification factor differs among genes, however, in this step, even if amplification bias occurs among genes or molecules, after data are obtained using a sequencer, the amplification bias can be corrected by utilizing the tag sequence for molecule discrimination, and therefore, quantitative data with high accuracy can be obtained. Incidentally, other amplification methods such as rolling circle amplification (RCA), NASBA, and LAMP methods can be used other than PCR amplification.

Here, a method for immobilizing the DNA probe 56 on the inside of the through-hole 55 of the pore array sheet 30 will be described. The surface of the through-hole 55 in the inside of the pore array sheet needs to be a surface on which the DNA probe 56 is immobilized at a high density and at the same time, nucleic acids such as mRNA and primers for PCR amplification, and proteins such as a reverse transcriptase and a polymerase are not adsorbed. Specifically, for example, a silane coupling agent for immobilizing the DNA probe 56 and a silanated MPC polymer for preventing adsorption are simultaneously immobilized in an appropriate ratio on the surface of the through-hole via a covalent bond, thereby realizing highly dense immobilization of the DNA probe 56 and stable prevention of adsorption of nucleic acids and proteins. Specifically, for example, first, a porous membrane made of alumina is immersed in an ethanol solution for 3 minutes. Then, a UVO3 treatment is performed for 5 minutes, followed by washing three times with ultrapure water. Subsequently, the membrane is immersed in an 80% ethanol solution containing 3 mg/ml of MPC_{0.8}-MPTMSi_{0.2} (MPC: 2-methacryloyloxyethyl phosphorylcholine, MPTMSi: 3-methacryloxypropyl trimethoxysilane) (see, Biomaterials 2009, 30: 4930-4938, and Lab Chip 2007, 7, 199-206) serving as a silanated MPC polymer having an average molecular weight of 9700 (polymerization degree: 40), 0.3 mg/ml of a silane coupling agent GTMSi (GTMSi: 3-glycidoxypropyltrimethoxysilane, Shin-Etsu Chemical Co., Ltd.), and 0.02% acetic acid serving as an acid catalyst for 2 hours. After washing the membrane with ethanol, the membrane is dried in a nitrogen atmosphere and subjected to a heat treatment in an oven at 120° C. for 30 minutes. Subsequently, in order to immobilize the DNA probe, 0.05 M borate buffer (pH 8.5) containing 1 μM of the DNA probe (SEQ ID NO: 1) modified with an amino group at the 5′ end, 7.5% glycerol, and 0.15 M NaCl is ejected onto the pore array sheet by the same technique as an inkjet printer so that the DNA probe containing the tag sequence for cell discrimination (1024 types) which differs for each region (25 μm×25 μm) is ejected at 100 pL/region. Thereafter, a reaction is allowed to proceed at 25° C. for 2 hours in a humidified chamber. Finally, in order to block unreacted glycide groups and remove excess DNA probes, the sheet is washed with a sufficient amount of a borate buffer (pH 8.5) containing 10 mM Lys, 0.01% SDS, and 0.15 M NaCl for 5 minutes. After this washing liquid is removed, the sheet is washed with 30 mM sodium citrate buffer containing 0.01% SDS and 0.3 M NaCl (2×SSC, pH 7.0) at 60° C. to remove excess DNA probes. In this manner, immobilization of the DNA probe 56 and the surface treatment are completed.

Hereinafter, a method for preparing a cDNA library pore array sheet and obtaining a gene expression profile using a next generation (large-scale) sequencer will be described. As one example, a pore array sheet (a portion including 100 regions) after capturing mRNA is introduced into a 0.2-mL tube. Separately, 58.5 μL of 10 mM Tris buffer (pH 8.0) containing 0.1% Tween 20, 4 μL of 10 mM dNTP, 22.5 μK of 5× RT buffer (SuperScript III, Invitrogen Corporation), 4 μL of 0.1 M DTT, 4 μL of RNaseOUT (Invitrogen Corporation), and 4 μL of Superscript III (reverse transcriptase, Invitrogen Corporation) are mixed, and the resulting mixture is dispensed into the above-mentioned tube containing the pore array sheet. Thereafter, the temperature of the solution and the pore array sheet is increased to 50° C. and maintained for 50 minutes to complete the reverse transcription reaction, whereby the first strand cDNA strand 59 having a complementary sequence to the mRNA is synthesized (FIG. 5(b)).

After the first strand cDNA strand 59 is synthesized, the reverse transcriptase is inactivated by maintaining the temperature at 85° C. for 1.5 minutes, followed by cooling to 4° C. After the solution is discharged, 0.2 mL of 10 mM Tris buffer (pH 8.0) containing RNase and 0.1% Tween 20 is dispensed into the tube containing the pore array sheet, thereby degrading mRNA, and then, the same amount of an alkaline denaturant is made to flow therein in the same manner, thereby removing and washing off the residue and the degraded products in the through-hole. Subsequently, after the solution was discharged, 69 μL of sterile water, 10 μL of 10× Ex Taq Buffer (TaKaRa Bio, Inc.), 10 μL of 2.5 mM dNTP Mix, 10 μL of a primer mix of 20 types of gene-specific sequences (SEQ ID NOS: 3 to 22, 10 μM each) to which a common sequence for PCR amplification (Reverse, SEQ ID NO: 2) has been added, and 1 μL of Ex Taq Hot start version (TaKaRa Bio, Inc.) are mixed, and the resulting mixture is dispensed into the tube. Thereafter, a reaction is allowed to proceed while maintaining the solution and the pore array sheet under the following conditions: 95° C. for 3 minutes→44° C. for 2 minutes→72° C. for 6 minutes, whereby a target gene-specific sequence primer 60 is annealed by using the first strand cDNA strand 59 as a template (FIG. 6(c)). Thereafter, a complementary strand elongation reaction is performed, whereby the second strand cDNA strand 61 is synthesized (FIG. 6(d)).

Subsequently, 49.5 μL of sterile water, 10 μL of 10× High Fidelity PCR Buffer (Invitrogen), 10 μL of 2.5 mM dNTP mix, 4 μL of 50 mM MgSO₄, 10 μL of 10 μM common sequence primer for PCR amplification (Forward, SEQ ID NO: 23), 10 μL of 10 μL common sequence primer for PCR amplification (Reverse, SEQ ID NO: 2), and 1.5 μL of Platinum Taq Polymerase High Fidelity (Invitrogen Corporation) are mixed. The solution present in the tube is removed, and immediately thereafter, the above-prepared solution is dispensed into the tube. Thereafter, the solution and the pore array sheet are maintained at 94° C. for 30 seconds, and then subjected to the following 3-step cycle: 94° C. for 30 seconds 55° C. for 30 seconds 68° C. for 30 seconds. The cycle is repeated 40 times. Finally, the solution and the pore array sheet are maintained at 68° C. for 3 minutes, and thereafter cooled to 4° C. In this manner, the PCR amplification step is performed (FIG. 6(e)). In order to realize such a thermal cycle, a heat block (an aluminum alloy or a copper alloy) with a heater and a temperature controller can be used. According to this, a region of interest of each of the 20 types of target genes is amplified, however, all PCR amplification products have substantially the same size of 200±8 bases. Then, the solution of the PCR amplification product accumulated in the solution in the inside and outside of the through-hole of the pore array sheet is collected. Purification is performed using PCR Purification Kit (QIAGEN, Inc.) for the purpose of removing free common sequence primers for PCR amplification (Forward/Reverse) and residual reagents such as enzymes contained in this solution. After the purified product is subjected to emPCR amplification or bridge amplification, an analysis is performed by subjecting the amplification product to a next generation sequencer.

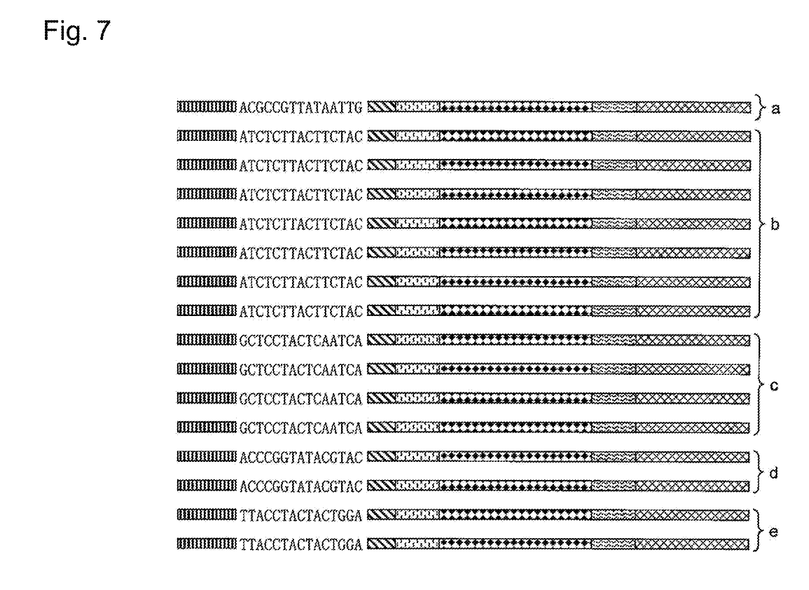

Next, a method for reducing amplification bias using the tag sequence for molecule discrimination will be described. In FIG. 7, a state where sequencing data are obtained as the same sequence other than the tag sequence for molecule discrimination is schematically shown (relevant portions of the obtained sequencing data are schematically shown by the same pattern). In FIG. 7, each of the reference numerals a, b, c, d, and e denotes the same sequence inclusive of the tag sequence for molecule discrimination which is a random sequence, and 1, 7, 4, 2, and 2 reads are obtained, respectively. These sequences are all one molecule at the time point when the second strand cDNA strand 61 is synthesized in FIG. 6(d), and the number of molecules is increased by the PCR amplification thereafter, and at the same time, the number of molecules becomes different. Therefore, the reads having the same tag sequence for molecule discrimination are derived from the same molecule and can be regarded as one molecule. As a result, the bias in the number of molecules for each sequence caused by PCR amplification after synthesizing the second strand cDNA strand 61 or adsorption on the inside of the pore array sheet that occurs when the solution is taken out to the outside is eliminated by regarding the reads having the same tag sequence for molecule discrimination as the same molecule as described above.

The pore array sheet prepared here can be used repeatedly, and with respect to a group of genes whose expression level is required to be known, a solution of a primer mix of target gene-specific sequences to which a common sequence primer for PCR amplification (Reverse, SEQ ID NO: 2) has been added is prepared, and in the same manner as described above, synthesis of the second strand cDNA strand, PCR amplification, and emPCR are performed, and then, an analysis may be performed using a sequencer. That is, by repeatedly using the cDNA library, highly accurate expression distribution measurement can be performed for necessary types of genes.

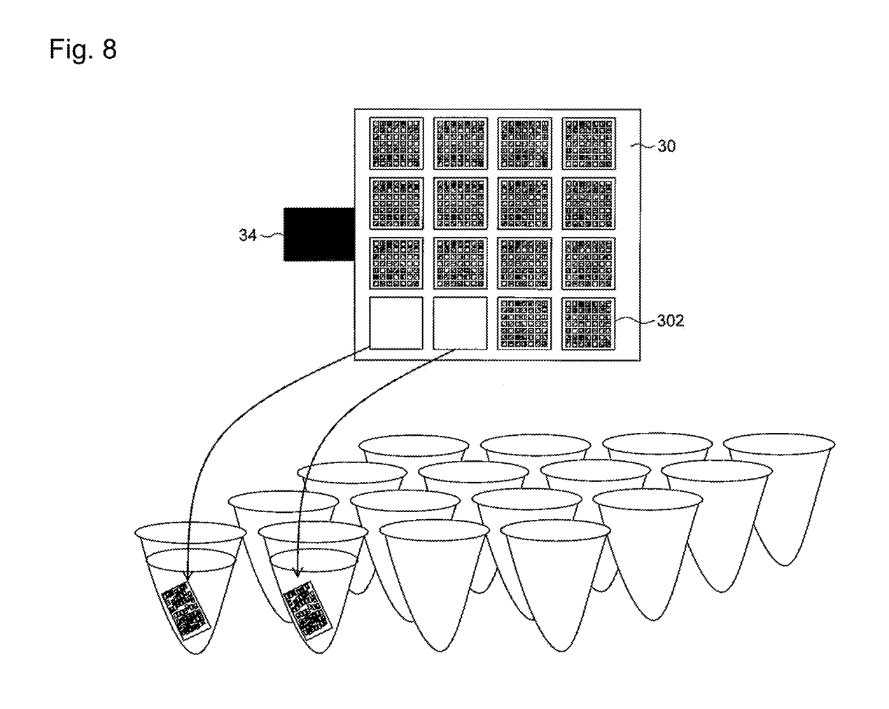

The size of the above-mentioned pore array sheet is 2×2 mm, and is a size capable of using a 0.2-mL tube as a reaction vessel, however, in general, the size of the pore array sheet may be made larger than 2×2 mm. However, in order to perform sample preparation shown in FIGS. 5 and 6 at this time, it is necessary to divide the pore array sheet and introduce the divided pore array sheet into the reaction vessel. This method will be described with reference to FIG. 8. On the pore array sheet 30, 16 chips 302 in which regions for capturing a biomolecule are arranged are formed. In order to separate the chips 302 by cutting, for example, the separation can be achieved by increasing the output power of the laser used for cell disruption to 10-fold.

Further, there are cases where after sample preparation is performed using a plurality of reaction vessels (in FIG. 8, 16 reaction vessels), sequencing is desired to be performed at a time. In such a case, a tag sequence for tube discrimination for discriminating a different reaction vessel based on the sequence information may be introduced into the target gene-specific sequence primer 60 to be used in the step of synthesizing the second strand cDNA strand in FIG. 6(c). The length of the tag sequence for tube discrimination may be determined according to the number of reaction vessels desired to be discriminated, however, in the case where the length of the tag sequence for tube discrimination is set to 5 bases in the same manner as the tag sequence for cell discrimination, ideally, 1024 vessels can be discriminated. Further, the position of inserting the tag sequence for tube discrimination can be determined to be between the gene-specific sequence hybridized to the first strand cDNA strand 59 and the primer for PCR amplification.

Further, in the above description, a region with a unit size of 2×2 mm is cut from the pore array sheet 30 and introduced into the reaction vessel, however, each region 301 corresponding to a single cell may be cut and introduced into the reaction vessel. In this case, the tag sequence for cell discrimination for discriminating a cell is omitted, and in place of this, only the tag sequence for tube discrimination may be used. It is a matter of course that both tag sequences may be used.

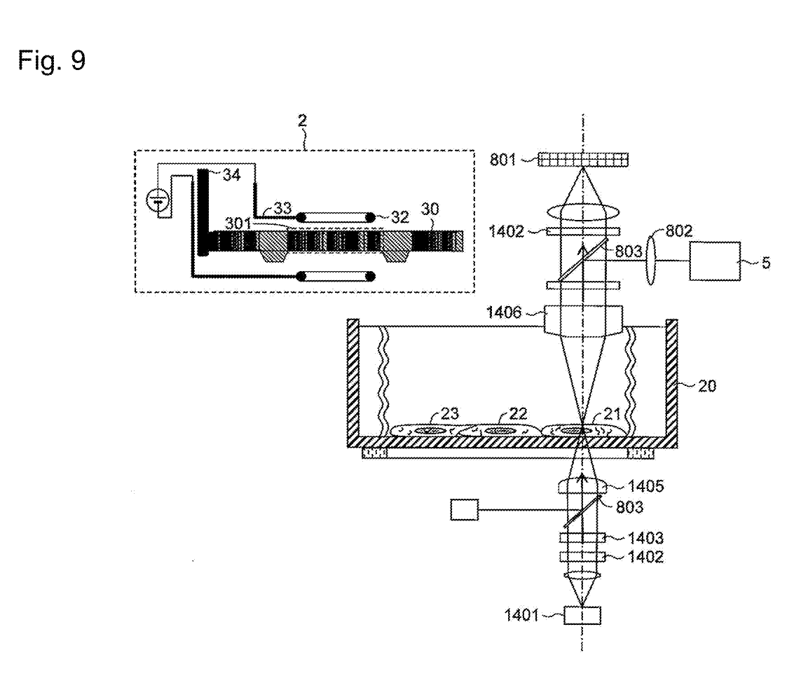

In the microscope system 1 in FIG. 1, an inverted fluorescence microscope is used, however, the fluorescence microscope may be replaced by a transmission differential interference contrast microscope. The structure of a system in such a case is shown in FIG. 9.

A differential interference contrast microscopic image is used only for observing the shape without using a fluorescent reagent, but is one of the measurement methods which have the smallest influence on cells when the cells should be returned in the body in regenerative medicine or the like. If a change in cell shape obtained from this image and a change in gene expression can be associated with each other, the system becomes a measurement system which can perform detailed cell classification with least damage to cells.

A biomolecule analyzer shown in FIG. 9 includes a light source 1401. In this example, the light source 1401 is a halogen lamp. The biomolecule analyzer shown in FIG. 9 further includes a polarizer 1402, a Wollaston filter 1403, a Wollaston prism 1404, a condenser lens 1405, an objective lens 1406, and an imaging device 801. As the imaging device 801, for example, a 1024×1024 pixel CCD camera can be used. In order to irradiate the center of the field of view of a differential interference contrast image with light from a laser light source for cell disruption 5 for disrupting a cell, a focus adjusting lens 802 and two dichroic mirrors 803 with an edge wavelength of 370 nm are disposed. The laser is on/off controlled by a control system (not shown), and is set so that a cell is irradiated with the laser only when needed. In this example, the pore array sheet 30 is movable, and in the case where a differential interference contrast image with high resolution is going to be obtained, the biomolecule collection system 2 is disposed outside the plate 20 as shown in FIG. 9, and then, an optical image is obtained and a cell to be measured is designated, and thereafter, the pore array sheet 30 is moved by using the XYZ stage 34 so as to bring the region 301 of the pore array sheet 30 close to the designated cell, and thereafter, the cell is disrupted.

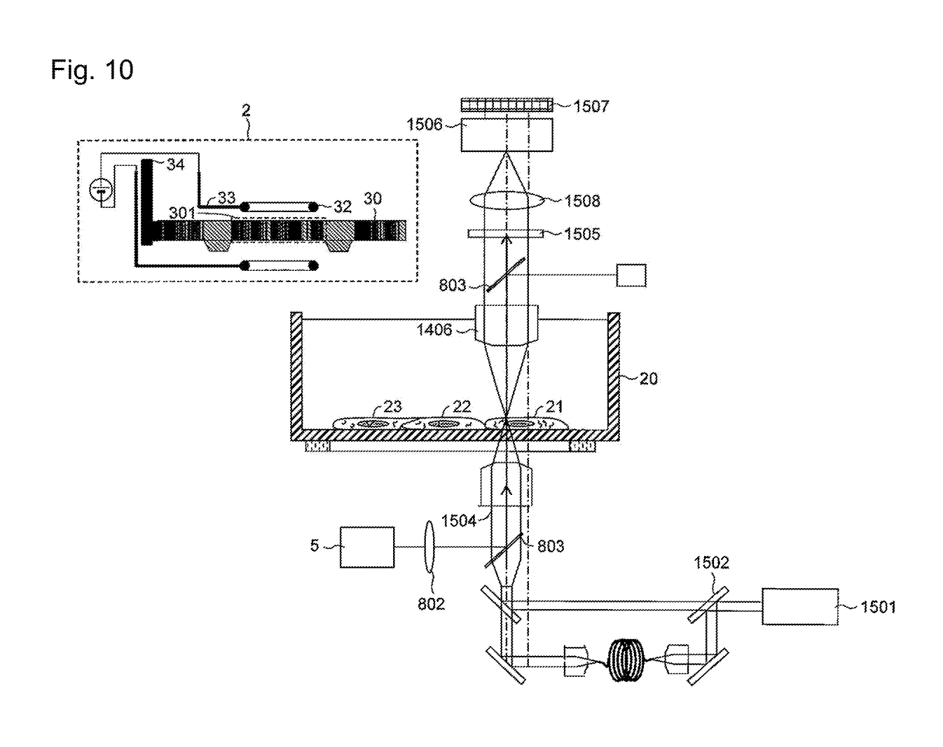

Further, as an example of the transmission microscope likewise, a CARS (Coherent anti-Stokes Raman scattering) microscope can also be used. In FIG. 10, the structural view of the device is shown. By using the CARS microscope, a spectrum corresponding to a chemical species in a laser-excited region can be obtained in the same manner as in the case of using a Raman microscope or an IR microscope, and therefore, the amount of information regarding a cell state can be increased as compared with a differential interference contrast microscope. However, CARS is a nonlinear process, and the signal intensity is higher than a Raman signal, and therefore, a sufficient signal can be obtained at a relatively low laser excitation intensity, and thus, it has an advantage that the damage to cells is low. According to such a device structure, the optical image obtained by the CARS microscope and the gene expression analysis data can be associated with each other, and the cell classification information based on the gene expression analysis can be provided for the measurement without using a label.

A biomolecule analyzer in FIG. 10 includes a light source 1501. Here, as the light source 1501, a pulse laser (microchip laser) is used. A laser is split into two components with a beam splitter 1502, and one light component is introduced into a nonlinear fiber (photonic crystal fiber) 1503 to produce a Stokes beam. The other light component is directly used as a pump beam and a probe beam, and collected on a sample (in cells 21, 22 and 23) with a water-immersion objective lens 1504 to produce an anti-Stokes beam. Only the anti-Stokes beam is transmitted with a high-pass filter 1505 and collected with an imaging lens 1508, and then, passed through a spectroscope 1506, and thus, a coherent anti-Stokes Raman spectrum is obtained with a spectroscopic CCD camera 1507. The configuration in which the center of an optical image obtained by CARS with light from the laser light source for cell disruption 5 for disrupting a cell is the same as in the case of the differential interference contrast microscope.

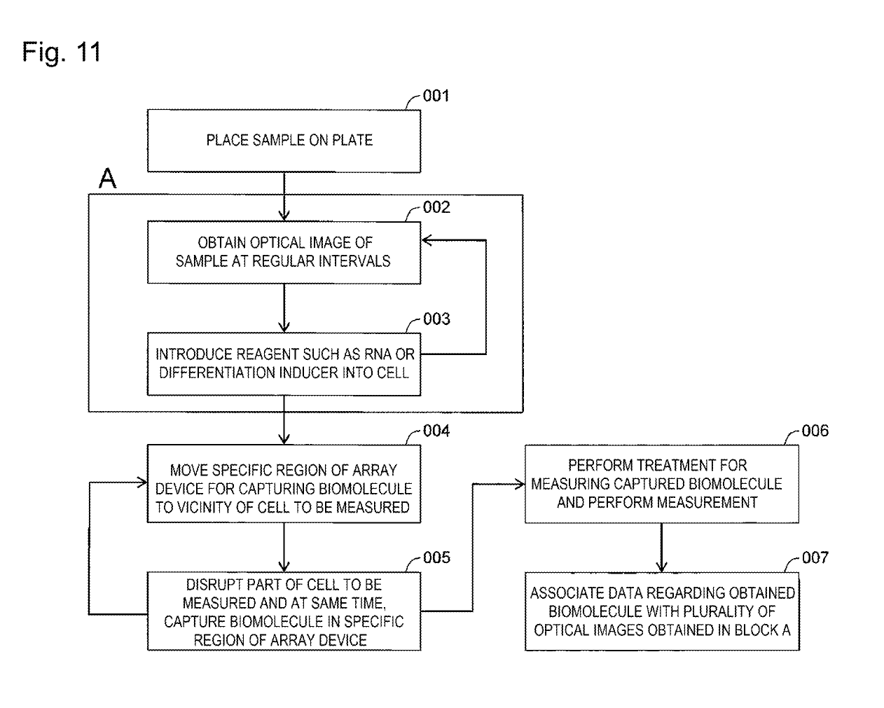

According to the present invention, a cell is specified by an optical image obtained with a fluorescence microscope or the like, and the gene expression data can be obtained by associating the data with the cell image. By utilizing this function, the dynamic characteristics of the cell can be confirmed with high accuracy. A flowchart for performing such an analysis is shown in FIG. 11. First, a cell sample is placed on a plate (step 001) and observed with the microscope, and an optical image is obtained (step 002). A reagent (RNA, a differentiation inducer, etc.) according to the purpose of the research is introduced into a cell (step 003), and a change of the cell on the optical image is confirmed. For this purpose, an optical image is obtained a plurality of times in some cases. At the time point when the association between the obtained optical image and the detailed cell state is desired to be confirmed, a specific region of the array device is moved to the vicinity of the cell selected by a user (step 004), the cell is disrupted, a biomolecule in the cell is captured on the array device (step 005), and the amount of the biomolecule is measured (step 006). By this quantitative determination of the biomolecule, a detailed cell state or a cell type is identified and associated with the optical image (step 007), whereby the optical image and the cell state or cell type can be associated with each other with high accuracy.

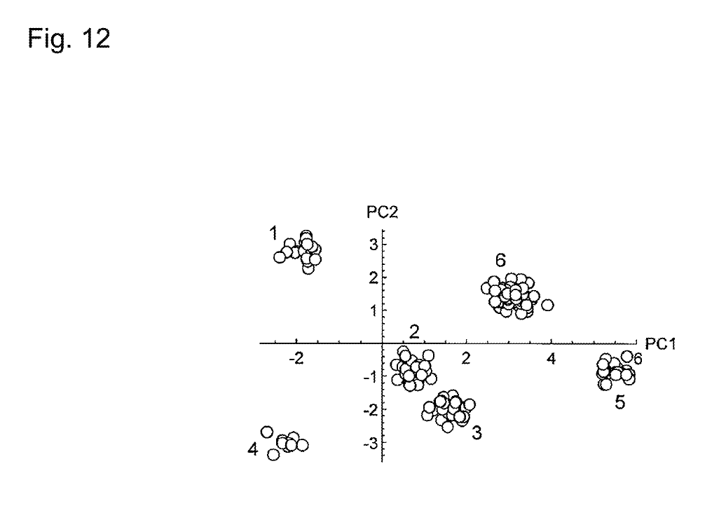

Next, a method for performing cell classification by optical imaging will be shown. After an optical image is obtained with a microscope, for example, a gene expression analysis is performed for 20 cells among 180 cells, and a principal component analysis is performed, and a view obtained by plotting the top two principal components is shown in FIG. 12. “PC” in the drawing is an abbreviation of “principal component”, and “PC1” refers to “first principal component”, and “PC2” refers to “second principal component”. The individual points correspond to the gene expression data of a single cell. In many cases, the data are divided into a plurality of clusters (6 clusters in this example) corresponding to the cell state or cell type. In FIG. 12, each point corresponds to a cell, and therefore, even if which cell is what type of cell cannot be determined only by the optical image, these can be associated with each other based on the gene expression analysis data. By utilizing this association, it is possible to cause a computer system to perform machine learning so as to determine what cell state or cell type when what kind of optical image is obtained, and after completion of the learning, it is possible to classify the cell state or cell type only by obtaining the optical image.

Incidentally, in this example, a principal component analysis is used for clustering based on the gene expression in cells, however, it is also possible to apply various methods such as hierarchical clustering and k-means clustering. In addition, as the method for machine learning, various methods to be used for data mining, such as a support vector machine are known, and any of these may be used.

### Second Embodiment

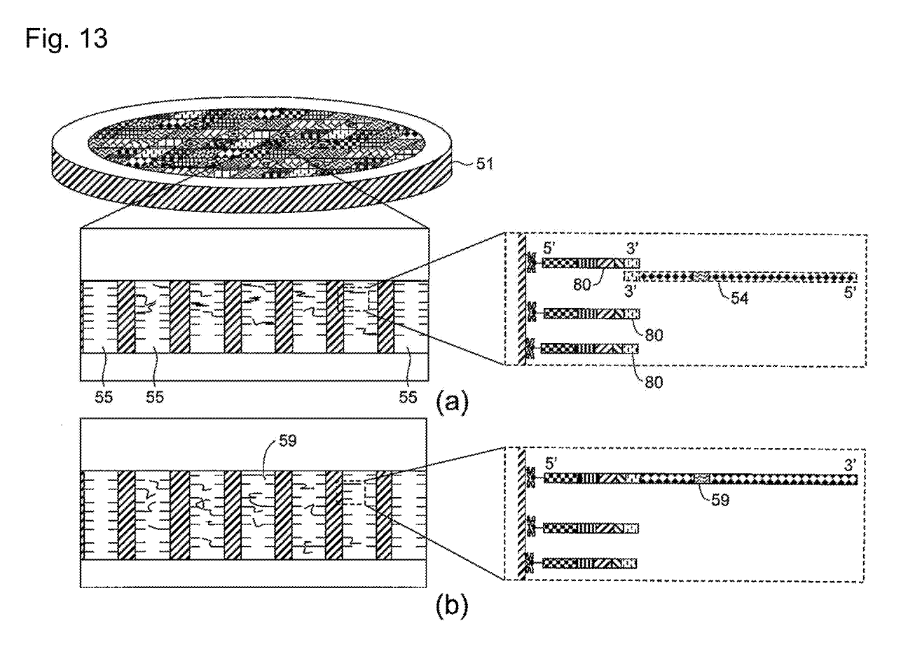

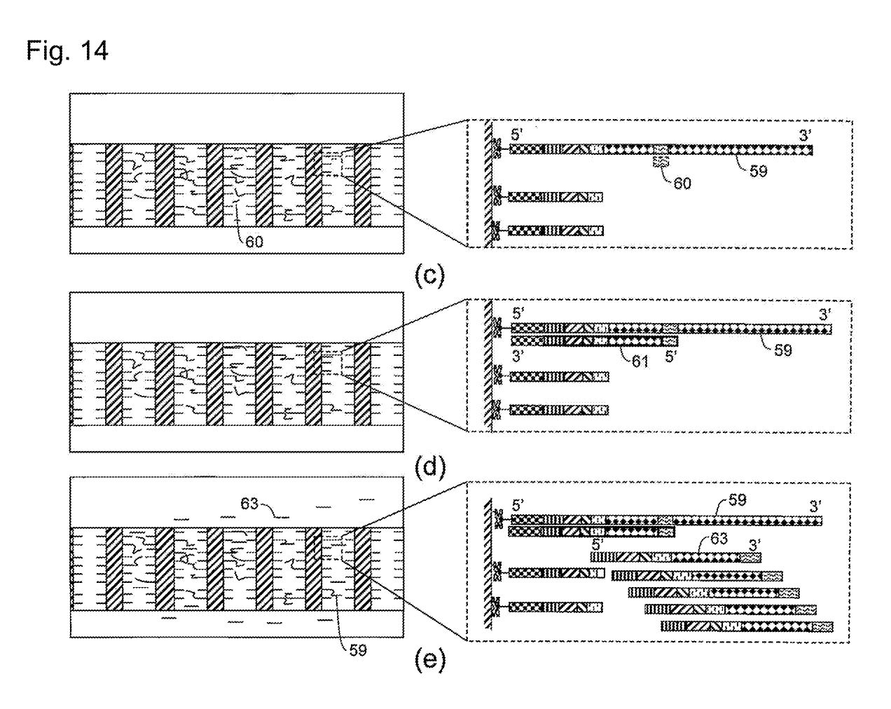

In this embodiment, an example in the case where a T7 promoter is used in place of PCR amplification will be described. A different point from the first embodiment is a method for preparing a sequencing sample. The procedure for sample preparation corresponding to FIGS. 5 and 6 is shown in FIGS. 13, 14, and 15. A DNA probe 80 immobilized on the inside of a pore array sheet 51 is composed of a T7 promoter sequence, a common sequence for emPCR amplification (Forward direction, SEQ ID NO: 24), a tag sequence for cell discrimination, a tag sequence for molecule discrimination, and an oligo(dT) sequence from the 5′ end side. By introducing the T7 promoter sequence into the DNA probe 80, a target sequence can be amplified in the subsequent step of amplifying cRNA 63 by IVT (In Vitro Transcription) (FIG. 14(e)). That is, the T7 promoter sequence is recognized by T7 RNA polymerase, and a transcription (amplification of cRNA 63) reaction is started from the sequence downstream of the T7 promoter sequence. In the same manner, by introducing the common sequence for PCR amplification, it can be used as a common primer in the subsequent emPCR amplification step. Further, by introducing the tag sequence for cell discrimination (composed of, for example, 5 bases) into the DNA probe 80, 4⁵ (=1024) single cells can be discriminated, which is the same as in the first embodiment. Further, by introducing the tag sequence for molecule discrimination (composed of, for examples, 15 bases) into the DNA probe 80, 4¹⁵ (=1.1×10⁹) molecules can be recognized, and therefore, it is possible to identify as to which molecule the enormous decoded data obtained using a next generation sequencer is derived from, which is also the same as in the first embodiment. That is, amplification bias among genes that occurs in the step of amplification by IVT/emPCR or the like can be corrected, and therefore, it becomes possible to quantitatively determine the level of mRNA originally present in the cell with high accuracy. The oligo (dT) sequence located closest to the 3′ end is hybridized to the poly A tail added to the 3′ end side of the mRNA, and therefore, utilized for capturing the mRNA (FIG. 13(a)).

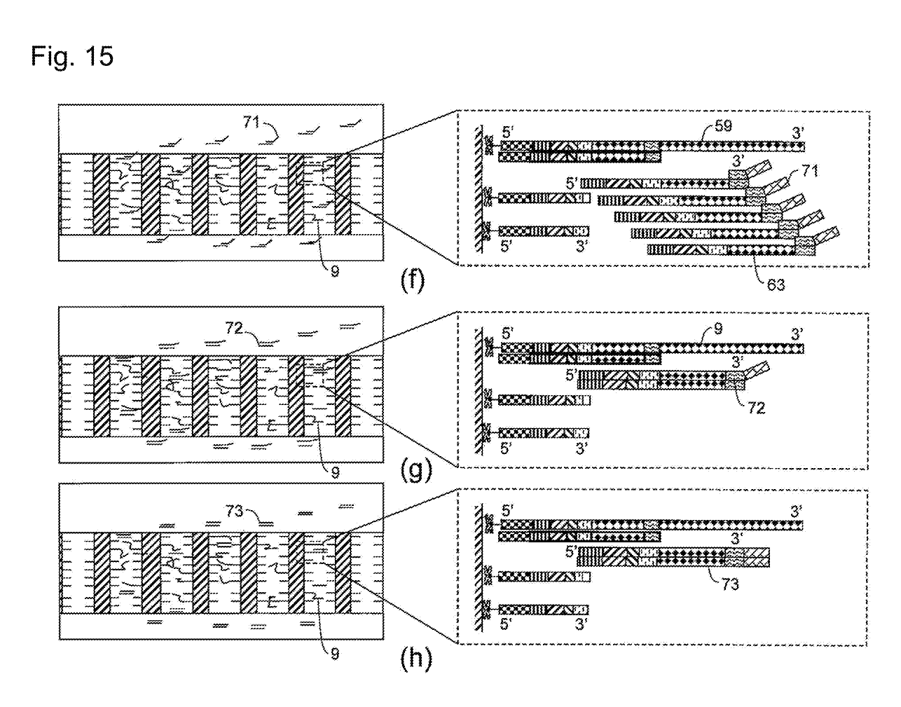

Next, the respective steps of the reaction will be sequentially described. As shown in FIG. 13(a), mRNA 54 is captured by an 18-base poly T sequence which is a complementary sequence to the poly A sequence at the 3′ end of the mRNA in the same manner as in the first embodiment. Subsequently, a first strand cDNA strand 59 is synthesized, whereby a cDNA library is constructed (FIG. 13(b)). Subsequently, a plurality (up to 100 types) of target gene-specific sequence primers 60 corresponding to the genes desired to be quantitatively determined are annealed to the first strand cDNA strand 59 (FIG. 14(c)), and by a complementary strand elongation reaction, a second strand cDNA strand 61 is synthesized (FIG. 14(d)). That is, the second strand cDNA strand 61 is synthesized under multiplex conditions. By doing this, with respect to each of the plurality of target genes, double-stranded cDNA having the common sequences for amplification (Forward/Reverse) at both ends, and containing the tag sequence for cell discrimination, the tag sequence for molecule discrimination, and the gene-specific sequence is synthesized. For example, 20 types (ATP5B, GAPDH, GUSB, HMBS, HPRT1, RPL4, RPLP1, RPS18, RPL13A, RPS20, ALDOA, B2M, EEF1G, SDHA, TBP, VIM, RPLP0, RPLP2, RPLP27, and OAZ1) of sequences, which are sequences composed of 20±5 bases in a region 109±8-bases upstream from the poly A tail of each of the target genes are used as the gene-specific sequences (SEQ ID NOS: 3 to 22). This is because the amplification products are made to have the same size of about 200 bases in the subsequence IVT amplification step. By making the amplification products have the same size, an effect that a complicated size fractionation purification step (electrophoresis→cutting out of gel→extraction and purification of amplification products) can be omitted, and the amplification products can be directly used for parallel amplification (emulsion PCR or the like) from one molecule is obtained. Subsequently, T7 RNA polymerase is introduced into the through-hole 55, and cRNA 63 is synthesized (FIG. 14(e)). By this process, about 1000 copies of cRNA 63 are synthesized. Further, in order to synthesize double-stranded DNA for emPCR, by using the amplified cRNA 63 as a template, a plurality (up to 100 types) of target gene-specific sequence primers 71 to which a common sequence for PCR amplification (Reverse) has been added are hybridized (FIG. 15(f)), whereby cDNA 72 is synthesized (FIG. 15(g)). Further, in the same manner as in the first embodiment, after the cRNA 63 is degraded using an enzyme, a second strand is synthesized using a forward common primer, whereby double-stranded DNA 73 for emPCR is synthesized (FIG. 15(h)). The obtained amplification products have the same size, and therefore can be directly subjected to emPCR and a next generation sequencer. In this step, even if amplification bias occurs among genes or molecules, after data are obtained by the next generation sequencer, the amplification bias can be corrected by utilizing the tag sequence for molecule discrimination, and therefore, quantitative data with high accuracy can be obtained, which is the same as in the first embodiment (see FIG. 7).

Next, a specific example of a series of steps will be described. After the first strand cDNA strand 59 is synthesized, the reverse transcriptase is inactivated by maintaining the temperature at 85° C. for 1.5 minutes, followed by cooling to 4° C. Thereafter, 10 mL of 10 mM Tris buffer (pH 8.0) containing RNase and 0.1% Tween 20 is injected from an inlet and discharged from an outlet, thereby degrading RNA, and then, the same amount of an alkaline denaturant is made to flow therein in the same manner, thereby removing and washing off the residue and the degraded products in the through-hole. Subsequently, 690 μL of sterile water, 100 μL of 10× Ex Taq Buffer (TaKaRa Bio, Inc.), 100 μL of 2.5 mM dNTP Mix, 100 μL of a primer mix of 20 types of gene-specific sequences (SEQ ID NOS: 3 to 22, 10 μM each) to which a common sequence for PCR amplification (Reverse, SEQ ID NO: 2) has been added, and 10 μL of Ex Taq Hot start version (TaKaRa Bio, Inc.) are mixed. The solution which fills the pore array sheet 51 is discharged from the outlet, and immediately thereafter, the above-prepared solution containing the reverse transcriptase is injected from the inlet. Thereafter, a reaction is allowed to proceed while maintaining the solution and the pore array sheet under the following conditions: 95° C. for 3 minutes→44° C. for 2 minutes→72° C. for 6 minutes, whereby the gene-specific sequence of the primer is annealed by using the first strand DNA strand 59 as a template (FIG. 14(c)). Thereafter, a complementary strand elongation reaction is performed, whereby the second strand cDNA strand 61 is synthesized (FIG. 14(d)).

Subsequently, 10 mL of 10 mM Tris buffer (pH 8.0) containing 0.1% Tween 20 is injected from the inlet and discharged from the outlet, thereby removing and washing off the residue and the degraded products in the through-hole. Further, 340 μL of sterile water, 100 μL of AmpliScribe 10× Reaction Buffer (EPICENTRE, Inc.), 90 μL of 100 mM dATP, 90 μL of 100 mM dCTP, 90 μL of 100 mM dGTP, 90 μL of 100 mM dUTP, 100 mM DTT, and 100 μL of AmpliScribe T7 Enzyme Solution (EPICENTRE, Inc.) are mixed. The solution which fills the pore array sheet is discharged from the outlet, and immediately thereafter, the above-prepared solution containing the reverse transcriptase is injected from the inlet. Thereafter, the temperature of the solution and the pore array sheet is increased to 37° C. and maintained for 180 minutes to complete a reverse transcription reaction, whereby cRNA amplification is performed. According to this, a region of interest of each of the 20 types of target genes is amplified, however, all cRNA amplification products have substantially the same size of 200±8 bases. The solution of the cRNA amplification product accumulated in the solution in the inside and outside of the through-hole is collected. Purification is performed using PCR Purification Kit (QIAGEN, Inc.) for the purpose of removing residual reagents such as enzymes contained in this solution, and the purified product is suspended in 50 μL of sterile water. In this solution, 10 μL of 10 mM dNTP mix and 30 μL of 50 ng/μL random primers are mixed. After the resulting mixture is heated to 94° C. for 10 seconds, the temperature of the mixture is decreased to 30° C. at 0.2° C./sec and the mixture is heated to 30° C. for 5 minutes, and then the temperature of the mixture is further decreased to 4° C. Thereafter, 20 μL of 5× RT buffer (Invitrogen Corporation), 5 μL of 0.1M DTT, 5 μL of RNaseOUT, and 5 μL of SuperScript III are mixed, and the resulting mixture is heated to 30° C. for 5 minutes, and the temperature of the mixture is increased to 40° C. at 0.2° C./sec. Purification is performed using PCR Purification Kit (QIAGEN, Inc.) for the purpose of removing residual reagents such as enzymes contained in this solution. After this purified product is subjected to emPCR amplification, an analysis is performed by subjecting the amplification product to a next generation sequencer.

### Third Embodiment

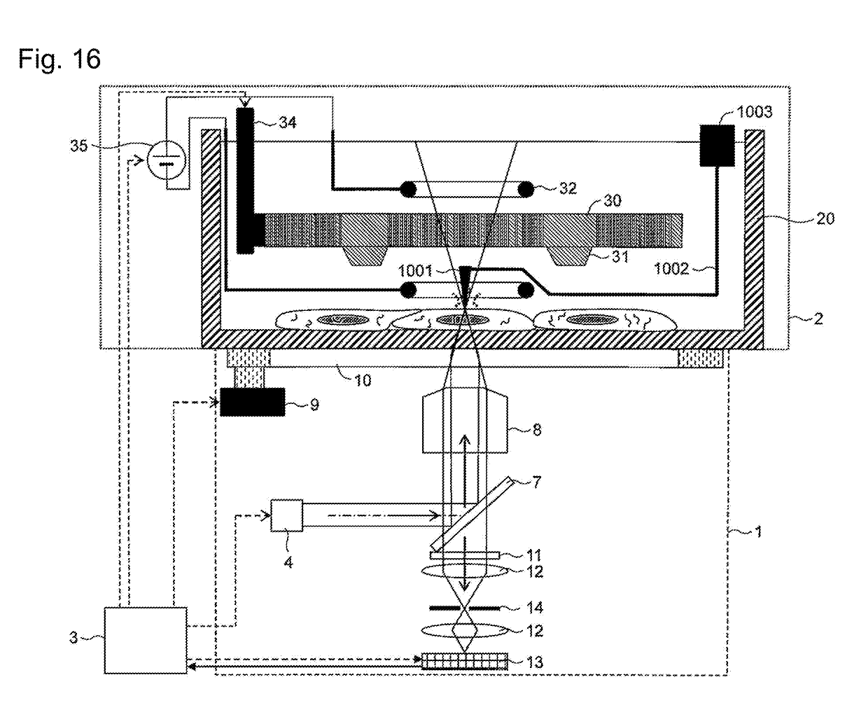

In the first and second embodiments, laser light is used as the unit which disrupts a cell, however, a needle composed of any of various materials may be used. In FIG. 16, the structural view of a device of this embodiment is shown. A needle 1001 is produced using a semiconductor process, and the length of the needle is, for example, 10 μm, and the diameter of a tip portion is set to 1 μm. As the material of a needle portion, a silicon oxide film can be used. It can be produced using the same production method as that for a cantilever of an atomic force microscope. As a holding member 1002 for the needle, a silicon substrate is used. This silicon substrate is driven by a Z stage 1003, and by piercing the cell membrane with the tip portion of the needle 1001, the cell membrane is perforated, and mRNA leaking out is guided to a pore array sheet 30 by electrophoresis. As for the position of the needle 1001 in the plane, the needle is disposed in the center of a platinum electrode 32 and fixed thereto. The needle 1001 may be replaced by a hollow needle, so that a biomolecule in the cell is released through the inside of the hollow needle.

Further, as the method for disrupting the cell by perforating the cell membrane, other than the above-mentioned method, a method of concentrating ultrasound, a method of concentrating charged particles or electron beams, or the like can be used. Incidentally, the number of cells to be disrupted with the needle or the like may be one or a few. Further, the cells may be perforated one by one, or a plurality of cells may be disrupted at a time. The method is selected according to the target sampling resolution.

### Fourth Embodiment

In this embodiment, a configuration for analyzing a peptide derived from a protein as a biomolecule is described. On the inside of a pore array sheet, an antibody which recognizes a protein or a peptide to be measured as the antigen is immobilized using a silane coupling agent instead of a DNA probe. The same immobilization conditions as described above may be used. The protein or peptide is not always negatively charged, and therefore, cannot be guided to the inside of the pore array sheet using electrophoresis. Accordingly, by using the pore array sheet as the plane of symmetry, a nozzle for sucking a solution is disposed on the opposite side to the cell to be measured. The solution sucked by the nozzle can be circulated by returning it to the inside of a plate. The inner diameter of the nozzle is, for example, 0.1 mm, and the suction rate can be set to 500 μL/sec. According to this, the flow of the solution is caused in the inside of the pore array sheet and in a region between the pore array sheet and the cell, and the biomolecule including a protein or a peptide released when the cell membrane is disrupted by a laser can be guided to the pore array sheet. Incidentally, the number of cells to be disrupted may be one or a few or so. The number of cells to be disrupted is selected according to the target sampling resolution.

After the biomolecule is captured in a specific region of the pore array sheet for each cell in this manner, the biomolecule is taken out from the plate, followed by drying. Then, 1 μL of sinapic acid at a saturated concentration is added thereto as a matrix agent for MALDI, and an analysis is performed in the same manner as common MALDI-TOF, whereby a single cell protein analysis with high sensitivity can be performed. Further, by performing the same treatment as described above without immobilizing an antibody specific to a specific biomolecule on the inside of the pore array sheet, it is also possible to analyze a protein or a peptide nonspecifically immobilized on the inner wall of the pore array sheet. In such a case, a single cell proteome analysis can be performed.

### Fifth Embodiment

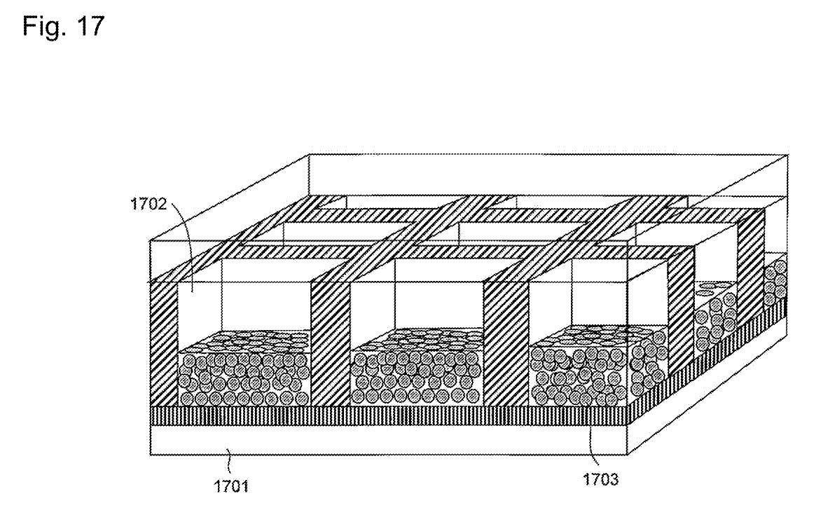

In this embodiment, a case where a bead array in which beads are packed on the surface is used as the array device in place of the pore array sheet composed of a porous membrane will be described. The configuration of a biomolecule analyzer is the same as that of the example in FIG. 1, but is different only in the point that a bead array as shown in FIG. 17 is used in place of the pore array sheet 30. In a bead array 1701, regions 1702 in which beads are held are arranged. The region 1702 corresponds to the region 301 in FIG. 1.

The bead array 1701 can be produced as follows. First, a porous membrane made of alumina used in the pore array sheet of the first embodiment is cut into a size of 2 mm×2 mm. Onto this sheet, a PDMS resin film with a thickness of 100 μm in which 50-μm square through-holes are formed at a pitch of 100 μm is bonded. Thereafter, a solution of beads 1703 having a DNA probe containing a tag sequence for cell discrimination which is different for each region 1702 immobilized thereon is injected by a piezo injector to be used for an inkjet printer. The solution is sucked into the porous membrane side by a capillary effect, followed by drying, and therefore, only the beads 1703 are packed. In one region 1702, 10⁴ to 10⁵ beads 1703 can be packed. Here, as the beads 1703, for example, magnetic beads with a diameter of 1 μm coated with streptavidin can be used. As the DNA probe, a DNA probe terminally modified with biotin is used and immobilized on the surface of the bead through streptavidin. Such beads are commercially available from a lot of manufacturers. The array device produced in this manner is placed facing down on an XYZ stage 34 such that the opening portions of the regions 1702 face the cells.

Note that the present invention is not limited to the above-mentioned embodiments, but includes various modifications. For example, with respect to part of the constituent elements of the embodiments, it is possible to perform addition, deletion, or replacement using other constituent elements.

All publications, patents, and patent applications cited herein are hereby incorporated by reference in their entirety.

### REFERENCE SINGS LIST

1 microscope system
2 biomolecule collection system
3 control system
4 laser light source for fluorescence microscope
5 laser light source for cell disruption
6 dichroic mirror
7 dichroic mirror
9 stage
10 sample stage
11 band-pass filter
12 imaging lens
13 optical receiver
14 pinhole
20 plate
21 adherent culture cell
22 adherent culture cell
23 adherent culture cell
30 pore array sheet
31 separation wall
32 platinum electrode
33 shield wire
34 XYZ stage
35 power supply
36 mRNA
39 reference electrode
41 power supply
42 quartz substrate
43 gold electrode
44 through-hole
51 pore array sheet
54 mRNA
55 through-hole
56 DNA probe
59 first strand cDNA strand
60 target gene-specific sequence primer
61 second strand cDNA strand
62 double-stranded cDNA
63 cRNA
71 target gene-specific sequence primer
72 cDNA
73 double-stranded DNA
80 DNA probe
301 region
302 chip
801 imaging device
802 focus adjusting lens
803 dichroic mirror
1001 needle
1002 holding member
1003 Z stage
1401 light source
1402 polarizer
1403 Wollaston filter
1404 Wollaston prism
1405 condenser lens
1406 objective lens
1501 light source
1502 beam splitter
1503 nonlinear fiber
1504 water-immersion objective lens
1505 high-pass filter
1506 spectroscope
1507 spectroscopic CCD camera
1508 imaging lens
1701 bead array
1702 region
1703 bead

1. A biomolecule analyzer, comprising:
a first unit configured to obtain an optical image of a plurality of cells;
a second unit configured to disrupt a part or the whole of at least one cell of the plurality of cells;
an array device in which regions for capturing a biomolecule in the cell released by the disrupting unit are arranged; and
a third unit configured to associate the region in which the biomolecule is captured in the array device with a portion corresponding to the cell disrupted by the disrupting unit in the optical image.

2. The biomolecule analyzer according to claim 1, further comprising a fourth unit configured to place the region of the array device and the cell to be disrupted in close proximity of each other before the cell is disrupted by the disrupting unit.

3. The biomolecule analyzer according to claim 1, further comprising a fifth unit configured to suck the biomolecule in the cell to be released into the region of the array device or allows the biomolecule to migrate into the region.

4. The biomolecule analyzer according to claim 1, wherein the array device is a porous membrane or a bead array having beads packed on the surface thereof.

5. The biomolecule analyzer according to claim 1, wherein a probe molecule for selectively capturing the biomolecule in the cell is immobilized on at least one of the surface and in the inside of the array device.

6. The biomolecule analyzer according to claim 5, wherein the biomolecule in the cell is mRNA, and the probe molecule is a DNA probe.

7. The biomolecule analyzer according to claim 6, wherein the DNA probe has a sequence which is different for each position of the array device.

8. The biomolecule analyzer according to claim 5, wherein the biomolecule in the cell is a protein or a peptide, and the probe molecule is an antibody.

9. The biomolecule analyzer according to claim 1, wherein the disrupting unit is a laser.

10. The biomolecule analyzer according to claim 1, wherein the disrupting unit is a needle.

11. The biomolecule analyzer according to claim 1, wherein the disrupting unit is a hollow needle, and the biomolecule in the cell is released through an interior portion of the hollow needle.

12. The biomolecule analyzer according to claim 1, wherein the disrupting unit is an electron beam or a charged particle beam.

13. The biomolecule analyzer according to claim 1, wherein the plurality of cells are three-dimensionally arranged in a gel.


SEQUENCE LISTING (24 sequences)

SEQ ID NO: 1 - DNA, 70 bp, Artificial
  synthetic
ccatctcatccctgcgtgtctccgactcagagctannnnnnnnnnnnnnnttttttttttttttttttvn

SEQ ID NO: 2 - DNA, 23 bp, Artificial
  synthetic
cctctctatgggcagtcggtgat

SEQ ID NO: 3 - DNA, 45 bp, Artificial
  synthetic
cctctctatgggcagtcggtgatccctaacccaaaaagcttcatt

SEQ ID NO: 4 - DNA, 45 bp, Artificial
  synthetic
cctctctatgggcagtcggtgatcactgaatctcccctcctcaca

SEQ ID NO: 5 - DNA, 43 bp, Artificial
  synthetic
cctctctatgggcagtcggtgatcgtttctggcctgggttttg

SEQ ID NO: 6 - DNA, 43 bp, Artificial
  synthetic
cctctctatgggcagtcggtgatgatgactgccttgcctcctc

SEQ ID NO: 7 - DNA, 48 bp, Artificial
  synthetic
cctctctatgggcagtcggtgattagtagtgtttcagtaatgttgact

SEQ ID NO: 8 - DNA, 43 bp, Artificial
  synthetic
cctctctatgggcagtcggtgataagaagcctgctgcataaac

SEQ ID NO: 9 - DNA, 49 bp, Artificial
  synthetic
cctctctatgggcagtcggtgataagtggaagcaaagaaagaagaatcc

SEQ ID NO: 10 - DNA, 41 bp, Artificial
  synthetic
cctctctatgggcagtcggtgatgtgtccgaggccagcaca

SEQ ID NO: 11 - DNA, 48 bp, Artificial
  synthetic
cctctctatgggcagtcggtgattctagaagcagaaatagactgggaa

SEQ ID NO: 12 - DNA, 48 bp, Artificial
  synthetic
cctctctatgggcagtcggtgatgagattgttaagcagattacttcca

SEQ ID NO: 13 - DNA, 42 bp, Artificial
  synthetic
cctctctatgggcagtcggtgatttgcccgcgctctttcttc

SEQ ID NO: 14 - DNA, 50 bp, Artificial
  synthetic
cctctctatgggcagtcggtgatattcatatttacttcttatacatttga

SEQ ID NO: 15 - DNA, 43 bp, Artificial
  synthetic
cctctctatgggcagtcggtgataaagccttcaatcagggcaa

SEQ ID NO: 16 - DNA, 41 bp, Artificial
  synthetic
cctctctatgggcagtcggtgatccagggagcgtggcactt

SEQ ID NO: 17 - DNA, 43 bp, Artificial
  synthetic
cctctctatgggcagtcggtgatctccagtattgcaggacaga

SEQ ID NO: 18 - DNA, 43 bp, Artificial
  synthetic
cctctctatgggcagtcggtgataatcttgtgctagaatactt

SEQ ID NO: 19 - DNA, 43 bp, Artificial
  synthetic
cctctctatgggcagtcggtgattcggacgaggatatgggatt

SEQ ID NO: 20 - DNA, 44 bp, Artificial
  synthetic
cctctctatgggcagtcggtgatatgagaagaaggaggagtctg

SEQ ID NO: 21 - DNA, 41 bp, Artificial
  synthetic
cctctctatgggcagtcggtgatgggaggccaaggtcaagt

SEQ ID NO: 22 - DNA, 45 bp, Artificial
  synthetic
cctctctatgggcagtcggtgatagaagtttcttatttggagtct

SEQ ID NO: 23 - DNA, 30 bp, Artificial
  synthetic
ccatctcatccctgcgtgtctccgactcag

SEQ ID NO: 24 - DNA, 112 bp, Artificial
  synthetic
aaacgacggccagtgaattgtaatacgactcactataggcgcccatctcatccctgcgtgtctccgactcagagctannnnnnnnnnnnnnnttttttttttttttttttvn